(19) United States
(12) Patent Application Publication
(10) Pub. No.: US 2020/0323574 A1
(43) Pub. Date: Oct. 15, 2020

(54) ORTHOPEDIC SCREWS AND CANNULATED ORTHOPEDIC SCREWS COMPRISING A TIP, A HEAD, AND A SHAFT THAT COMPRISES A THREAD HAVING A PLURALITY OF GROOVES

(71) Applicant: GARY A. ZWICK, TRUSTEE OF THE EVEREST TRUST UTA APRIL 20, 2017, Cleveland, OH (US)

(72) Inventors: George J. PICHA, Brecksville, OH (US); Grant Wesley PHILLIPS, Richfield, OH (US); Rachel SMITH, Brecksville, OH (US); James PRICE, Stow, OH (US)

(21) Appl. No.: 16/493,926

(22) PCT Filed: Mar. 13, 2018
(86) PCT No.: PCT/US18/22138

§ 371 Date: Sep. 13, 2019

Related U.S. Application Data
(60) Provisional application No. 62/470,546, filed on Mar. 13, 2017.

(51) Int. Cl. A61B 17/86; A61B 17/88

(52) U.S. Cl. CPC A61B 17/863; A61B 17/864; A61B 17/1655; A61B 17/8605; A61B 17/8897; A61B 17/8635

(57) ABSTRACT

Orthopedic screws are provided that include a tip, a head, and a shaft extending between the tip and the head. The shaft comprises a shaft core and at least one thread. The thread is disposed helically along the shaft, extends radially from the shaft, and has a plurality of grooves oriented transversely with respect to the thread. The plurality of grooves define a series of thread segments and thread slots along the thread. The shaft core is solid from the tip to the head. Cannulated orthopedic screws also are provided that include a shaft core that is hollow from the tip to the head and that defines a lumen extending from a tip opening to a head opening. Methods of use of the orthopedic screws and the cannulated orthopedic screws also are provided.

### CROSS-REFERENCE TO RELATED APPLICATIONS

This application claims the benefit of U.S. Provisional Application No. 62/470,546, filed Mar. 13, 2017, the entire disclosure of which is hereby incorporated herein by reference.

### FIELD OF THE INVENTION

The invention relates to orthopedic screws and cannulated orthopedic screws, and more particularly to orthopedic screws and cannulated orthopedic screws that include a tip, a head, and a shaft extending between the tip and the head, wherein: (i) the shaft comprises a shaft core and at least one thread; (ii) the at least one thread is disposed helically along the shaft, extends radially from the shaft, and has a plurality of grooves oriented transversely with respect to the at least one thread; (iii) the plurality of grooves define a series of thread segments and thread slots along the at least one thread; and (iv) the shaft core is solid from the tip to the head.

### BACKGROUND OF THE INVENTION

Orthopedic screws, including cannulated orthopedic screws, are hard-tissue implants that can be used to fix bone or bone fragments relative to each other and/or to position other hard-tissue implants with respect to bone. Many types of orthopedic screws have been disclosed.

For example, Prien et al., U.S. Pat. No. 9,198,701, discloses a bone fastener for use in orthopedic surgery for anchoring an intramedullary nail to bone. The bone fastener has a shaft with a front region and a rear region. The front region has an anchoring thread for attaching the bone fastener to bone. The front region has a first core diameter and the anchoring thread has a thread pitch. The rear region has one or more explantation grooves helically arranged at a pitch substantially corresponding to the pitch of the anchoring thread for facilitating explantation of the bone fastener. Two axially spaced apart grooves or groove sections are separated by a flat shaft portion defining an outside diameter of the rear region. The rear region has a second core diameter greater than the first core diameter.

Also for example, Christen et al., U.S. Pub. No. 2010/0211118, discloses a self-tapping bone screw for use as a compression screw or a locking screw for an implant. The self-tapping bone screw has a screw shank, which has a front tip, a cutting region, an intermediate region, and a rear head region. A thread extends, in a threaded portion of the screw shank, over a transition region comprising mutually adjoining parts of at least the cutting region and the intermediate region. An outside diameter and a root diameter of the screw shank are defined by the thread in the threaded region. In the transition region the root diameter of the screw shank in the cutting region is greater than the root diameter of the screw shank in the intermediate region. Furthermore, in the transition region the outside diameter of the screw shank is constant.

General purpose screws can be used for fixation of workpieces generally. Many types of general purpose screws also have been disclosed.

For example, Hsu, U.S. Pat. No. 6,789,991, discloses a screw that is shaped to be screwed into a workpiece easily, without causing cracks on the workpiece, while the tightness of connection is not reduced. The screw includes a rod-shaped portion, and a spiral-shaped thread winding around the rod-shaped portion. Several cutting trenches are formed on the rod-shaped portion for cutting bits from a workpiece when the screw is being screwed into the workpiece. A tapering front end portion of the rod-shaped portion is provided with a notch, and cutting teeth are provided on that portion of the thread corresponding to the tapering front end portion.

Also for example, Lajewardi et al., U.S. Pat. No. 9,581,183, discloses a multi-thread screw. The screw includes a head end, a shank and a tapered end. The head end includes a tool engaging part. The head end is located at one end of the shank and the tapered end is located at an opposite end of the shank. Multiple threads are formed along the screw including a high thread, a low thread and a set of intermediate threads.

Unfortunately orthopedic screws are not generally designed to both provide immediate load transfer upon implantation and prevent stress shielding over time. This can result in delayed healing of bone following implantation of an orthopedic screw and/or result in loosening of the orthopedic screw with respect to bone over time. Moreover, general purpose screws are not generally designed for orthopedic applications at all. Accordingly, a need exists for improved orthopedic screws, including cannulated orthopedic screws.

### SUMMARY

An orthopedic screw is provided. The orthopedic screw includes a tip, a head, and a shaft extending between the tip and the head. The shaft comprises a shaft core and at least one thread. The at least one thread is disposed helically along the shaft, extends radially from the shaft, and has a plurality of grooves oriented transversely with respect to the at least one thread. The plurality of grooves define a series of thread segments and thread slots along the at least one thread. The shaft core is solid from the tip to the head.

A method of use of an orthopedic screw for fixation of a hard tissue in an individual in need thereof also is provided. The orthopedic screw is as described above. The method comprises a step of rotationally driving the orthopedic screw into the hard tissue of the individual.

A cannulated orthopedic screw also is provided. The cannulated orthopedic screw comprises a tip having a tip opening, a head having a head opening, and a shaft extending between the tip and the head. The shaft comprises a shaft core and at least one thread. The at least one thread is disposed helically along the shaft, extends radially from the shaft, and has a plurality of grooves oriented transversely with respect to the at least one thread. The plurality of grooves define a series of thread segments and thread slots along the at least one thread. The shaft core is hollow from the tip to the head and defines a lumen extending from the tip opening to the head opening.

A method of use of a cannulated orthopedic screw for fixation of a hard tissue in an individual in need thereof also is provided. The cannulated orthopedic screw is as described above. The method comprises a step of rotationally driving the cannulated orthopedic screw into the hard tissue of the individual.

### BRIEF DESCRIPTION OF THE DRAWINGS

These and other features, aspects, and advantages of the present disclosure are better understood when the following detailed description is read with reference to the accompanying drawings, in which:

FIG. 1 is a side view of an orthopedic screw as disclosed herein;

FIG. 2 is an enlarged side view of a portion of the orthopedic screw of FIG. 1;

FIG. 3 is a side view of another orthopedic screw as disclosed herein;

FIG. 4 is an enlarged side view of a portion of the orthopedic screw of FIG. 3;

FIG. 5 shows bottom perspective views of portions of orthopedic screws having slot depths of (A) 250 μm, (B) 500 μm, and (C) 750 μm.

FIG. 6 is a further enlarged side view of a portion of the orthopedic screw of FIG. 3;

FIG. 7 is a perspective view of an orthopedic screw corresponding to a trochanteric screw;

FIG. 8 is a bottom view of the trochanteric screw of FIG. 7;

FIG. 9 is a first side view of the trochanteric screw of FIG. 7;

FIG. 10 is a second side view of the trochanteric screw of FIG. 7;

FIG. 11 is another perspective view of the trochanteric screw of FIG. 7;

FIG. 12 is a side view of a cannulated orthopedic screw as disclosed herein;

FIG. 13 is a perspective view of a cannulated orthopedic screw corresponding to a cannulated trochanteric screw;

FIG. 14 is a bottom view of the cannulated trochanteric screw of FIG. 13;

FIG. 15 is a first side view of the cannulated trochanteric screw of FIG. 13;

FIG. 16 is a second side view of the cannulated trochanteric screw of FIG. 13;

FIG. 17 is another perspective view of the cannulated trochanteric screw of FIG. 13;

FIG. 18 is a top view of the cannulated trochanteric screw of FIG. 13; and

FIG. 19 is a sectional view of the cannulated trochanteric screw of FIG. 14.

### DETAILED DESCRIPTION

As set forth in the figures, example orthopedic screws and cannulated orthopedic screws are provided. The orthopedic screws and cannulated orthopedic screws provide advantages, including for example that the orthopedic screws and cannulated orthopedic screws can promote hard-tissue remodeling and growth of the hard tissue at the site of implantation and that the interface of the orthopedic screws or cannulated orthopedic screws and the hard tissue can withstand substantial yield/elongation and load before failure. Without wishing to be bound by theory, it is believed that these advantages are based on properties of the orthopedic screws and cannulated orthopedic screws and the interface resulting from implantation thereof.

This is because the interface can have a continuous phase corresponding to the hard tissue and a discontinuous phase corresponding to the orthopedic screws or cannulated orthopedic screws. The hard tissue can also make up at least 40% of the volume of the interface, and the product of the Young's modulus of elasticity of the hard tissue and the volume of the tissue and the product of the Young's modulus of elasticity of the screws and the volume of thread segments of the screws can be well matched. Thus, the interface can exhibit mechanical properties similar to those of the bulk hard tissue adjacent to the interface. Also, the thread segments may be driven into the hard-tissue, e.g. based on rotationally driving the screws during implantation, potentially eliminating micro-motion and migration of the screws over time. In addition, the screws may promote rich vascularization of the hard tissue of the interface, enhancing wound healing, providing nutritional support, accelerating healing, remodeling, and integration of the hard tissue, and limiting the potential for infection of the hard tissue. Rapid or immediate integration of the hard tissue into the space between the thread segments of the screws may also prevent detrimental cellular reactions at the interface, such as formation of fibrous tissue, seroma, or thrombosis.

It is believed that implantation of the orthopedic screws and cannulated orthopedic screws will result in the thread segments of the screws contacting the hard tissue. The thread segments may penetrate the hard tissue, e.g. partially or completely, upon implantation of the screws. In such cases, the screws can provide immediate load transfer upon implantation and prevent stress shielding over time, thus promoting hard-tissue remodeling and growth at the site of implantation. Alternatively or additionally, in some cases the thread segments may penetrate the hard tissue later, under physiological loading. Also alternatively or additionally, over time the hard tissue may grow in and around the thread segments, thus occupying thread slots between the thread segments, e.g. during healing.

The interface resulting from implantation of the orthopedic screws and cannulated orthopedic screws into the hard tissue will be, or can become, an interface that is continuous with respect to the hard tissue and discontinuous with respect to the screws. Such an interface will further exhibit properties similar to those of the bulk hard tissue adjacent to the interface, e.g. high resilience to load. The result is that the interface following implantation of orthopedic screws and cannulated orthopedic screws into a hard tissue is surprisingly long-lasting and resilient to load.

It is believed that this will be particularly beneficial for use in people having bone with a low bone mass and/or a deteriorated bone structure, due for example to one or more of age-related osteoporosis, idiopathic primary osteoporosis, secondary osteoporosis, or a neoplastic condition of the musculoskeletal system.

As used herein, the term “orthopedic screw” means a screw suitable for implantation into a hard tissue, for example in methods of fixation of a hard tissue in an individual in need thereof. Exemplary orthopedic screws include cortical bone screws, which can have a fine pitch, and cancellous bone screws, which can have a coarse pitch, among others.

As used herein, the term “cannulated orthopedic screw” means an orthopedic screw that has a lumen extending therethrough, such that the cannulated orthopedic screw can be introduced to a hard tissue over a guide wire or a guide pin. Exemplary cannulated orthopedic screws include cannulated trochanteric screws, cannulated pedicle screws (e.g. cannulated posterior cervical pedicle screws and cannulated posterior lumbar pedicle screws), and cannulated trauma plate screws.

Exemplary hard tissues suitable for implantation of the orthopedic screws and cannulated orthopedic screws include bone, cartilage, calcified cartilage, non-calcified cartilage, and tissue that has become mineralized. Exemplary hard tissues also include long bone, maxillary bone, mandibular bone, and membranous bone. Exemplary hard tissues also include tibia, femur, shoulder, small joints, skull, and metatarsal. Exemplary hard tissues also include spine. Exemplary hard tissues also include bones of the foot/ankle in addition to metatarsal, including talus, navicular, cuneiform bones, and phalanges.

As used herein, the term “interface resulting from implantation of the orthopedic screws and/or cannulated orthopedic screws into a hard tissue,” or more simply “interface,” means the product of implantation wherein the thread segments of the screws are contacting a hard tissue and the thread slots (and if present, channels) of the screws are occupied, partially or completely, by the hard tissue. The interface includes the thread segments, hard tissue that occupies the thread slots (and if present, the channels) of the screws, and any remaining unoccupied space in the thread slots (and if present, the channels).

In some example embodiments, e.g. immediately after implanting the orthopedic screw or cannulated orthopedic screw with at least some penetration of the thread segments into the hard tissue and/or after at least some remodeling and growth of the hard tissue to partially fill in space between the screw and the hard tissue, the thread segments are contacting the hard tissue, and the thread slots are partially occupied by the hard tissue. In other example embodiments, e.g. immediately after implanting the orthopedic screw or cannulated orthopedic screw with extensive penetration of the thread segments into the hard-tissue and/or after extensive remodeling and growth of the hard tissue to fill in all space between the screw and the hard tissue, the thread segments are contacting the hard tissue, and the thread slots are completely occupied by the hard tissue. In other example embodiments, the thread segments contact the hard tissue over time, based on remodeling and growth of hard tissue in and around the thread segments, e.g. during healing.

As used herein, the term “continuous,” when used for example in reference to the hard-tissue of an interface, means that the hard tissue forms a single continuous phase, extending throughout and across the interface. As used herein, the term “discontinuous,” when used for example in reference to the orthopedic screw or cannulated orthopedic screw of an interface, means that the screw does not form such a single continuous phase.

### Orthopedic Screws

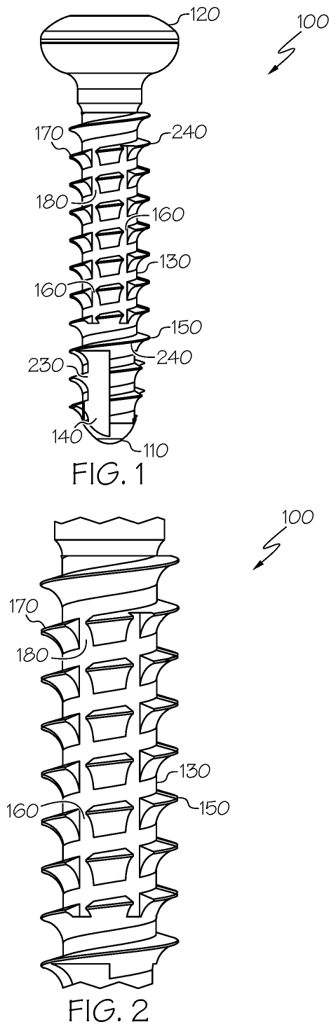

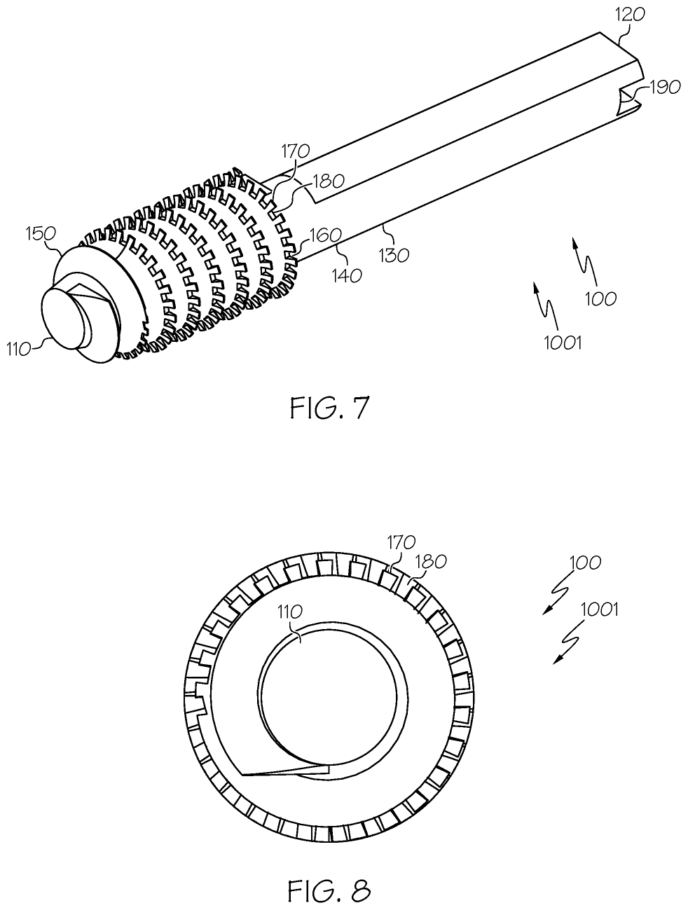

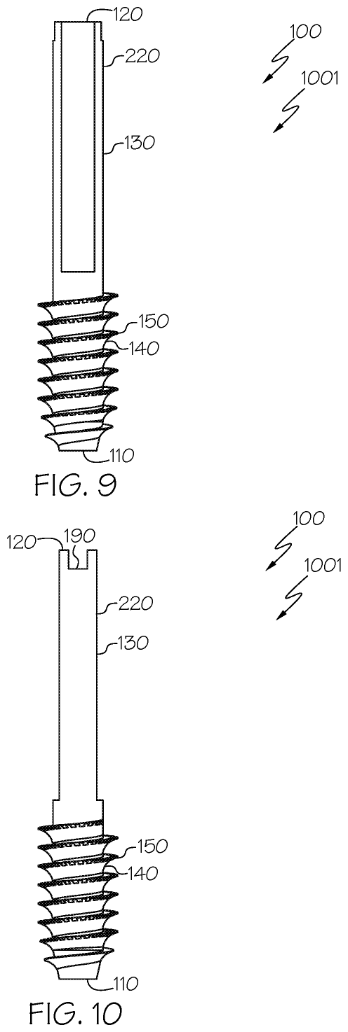

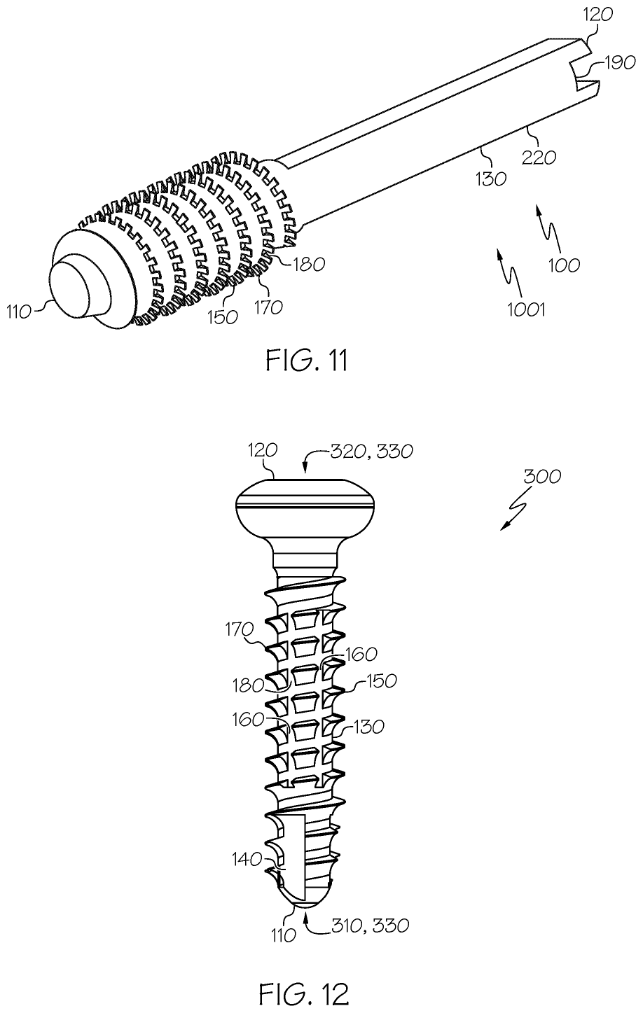

Considering the features of an example orthopedic screw in more detail, FIGS. 1-6 provide illustrations in various views of an example orthopedic screw 100, and FIGS. 7-11 provide illustrations in various views of an example orthopedic screw 100 corresponding to a trochanteric screw 1001.

As shown in FIG. 1, the orthopedic screw 100 includes a tip 110, a head 120, and a shaft 130 extending between the tip 110 and the head 120. The shaft 130 comprises a shaft core 140 and at least one thread 150. The at least one thread 150 is disposed helically along the shaft 130, extends radially from the shaft 130, and has a plurality of grooves 160 oriented transversely with respect to the at least one thread 150. The plurality of grooves 160 define a series of thread segments 170 and thread slots 180 along the at least one thread 150. The shaft core 140 is solid from the tip 110 to the head 120.

By the shaft core 140 being solid from the tip 100 to the head 120, it is meant that the shaft core 130 does not include hollow portions internal thereto. This can promote structural integrity of the orthopedic screw 100 despite the plurality of grooves 160.

As shown in FIG. 1, in some examples the orthopedic screw 100 is tapered from the shaft 130 to the tip 110. In other examples, the orthopedic screw 100 is not so tapered.

As shown in FIG. 7, in some examples (including, for example, the trochanteric screw 1001, among others) the head 120 comprises a tool engaging portion 190 for rotationally driving the orthopedic screw 100. In other examples, the head 120 does not comprise a tool engaging portion.

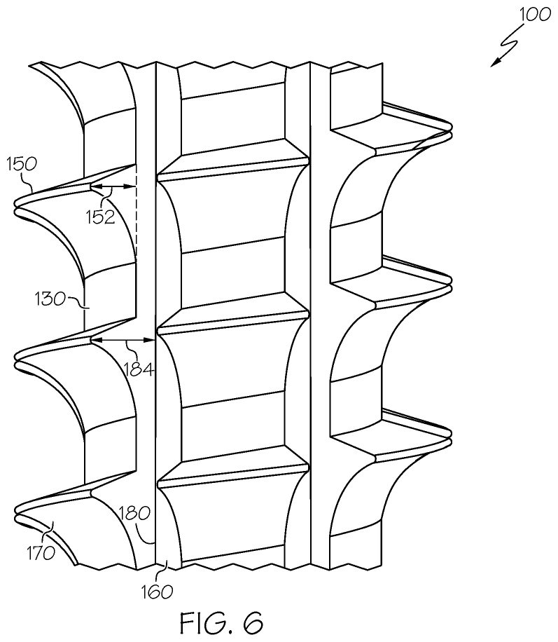

As shown in FIG. 6, in some examples at least one thread 150 has a thread height 152 of 100 μm to 10,000 μm. The thread height 152 corresponds to the distance between a minimal diameter of the thread 150 and a maximal diameter of the thread 150. The minimal diameter of the thread 150 can be defined by the circumference of the shaft 130 at a position at which the thread 150 extends radially from the shaft 130. The maximal diameter of the thread 150 can be defined by the thread 150 at a furthest point at which the thread 150 extends radially from the shaft 130. As will be appreciated, the thread height 152 can vary along the orthopedic screw 100, depending for example on dimensions of the shaft 130 and the thread 150 along the orthopedic screw 100. For example, the thread height 152 can vary along the orthopedic screw 100 in accordance with loads that the orthopedic screw 100 will need to carry and/or hard tissue with which the orthopedic screw 100 will interface. In some examples, the thread 150 has a thread height 152 of 120 μm to 5,000 μm, 150 μm to 1,000 μm, 200 μm to 800 μm, 250 μm to 600 μm, 300 μm to 500 μm, or about 400 μm. Also in some examples, the thread 150 has a thread height 152 of 100 μm to 500 μm, 300 μm to 1,000 μm, 500 μm to 5,000 μm, or 1,000 μm to 10,000 μm.

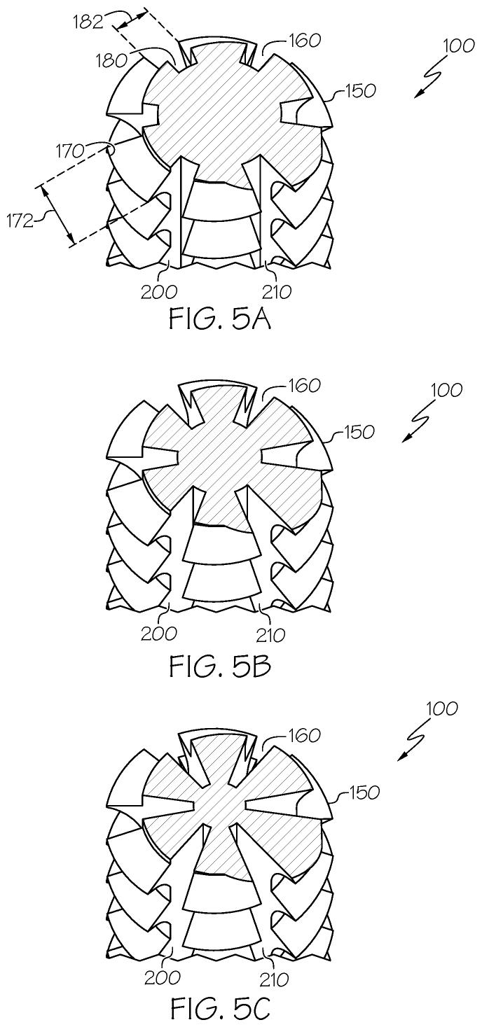

As shown in FIG. 5, in some examples the thread segments 170 have a thread segment width 172, measured as an arcuate length with respect to the shaft 130, of 100 μm to 10,000 μm. The thread segment width 172 can be measured along the thread 150 at a furthest point at which the thread 150 extends radially from the shaft 130. The arcuate length can be measured with respect to a center line along a major axis of the shaft 130. As will be appreciated, the thread segment width 172 can vary along the orthopedic screw 100, depending for example on dimensions of the shaft 130, the thread 150, and the plurality of grooves 160 along the orthopedic screw 100. In some examples, the thread segments 170 have a thread segment width 172 of 120 μm to 5,000 μm, 150 μm to 1,000 μm, 200 μm to 800 μm, 250 μm to 600 μm, 300 μm to 500 μm, or about 400 μm. Also in some examples, the thread segments 170 have a thread segment width 172 of 100 μm to 500 μm, 300 μm to 1,000 μm, 500 μm to 5,000 μm, or 1,000 μm to 10,000 μm.

As shown in FIG. 5, in some examples the thread slots 180 have a thread slot width 182, measured as an arcuate length with respect to the shaft 130, of 100 μm to 10,000 μm. The thread slot width 182 can be measured analogously to the thread segment width 172, along the thread 150 at a furthest point at which the thread 150 extends radially from the shaft 130. The arcuate length can be measured with respect to a center line along a major axis of the shaft 130. As will be appreciated, the thread slot width 182 can vary along the orthopedic screw 100, depending for example on dimensions of the shaft 130, the thread 150, and the plurality of grooves 160 along the orthopedic screw 100. In some examples, the thread slots 180 have a thread slot width 182 of 120 μm to 5,000 μm, 150 μm to 1,000 μm, 200 μm to 800 μm, 250 μm to 600 μm, 300 μm to 500 μm, or about 400 μm. Also in some examples, the thread slots 180 have a thread slot width 182 of 100 μm to 500 μm, 300 μm to 1,000 μm, 500 μm to 5,000 μm, or 1,000 μm to 10,000 μm.

As shown in FIG. 6, in some examples the thread slots 180 have a thread slot depth 184 of 100 μm to 10,000 μm. The thread slot depth 184 corresponds to the distance between a minimal diameter of the thread slot 180 and a maximal diameter of the thread slot 180. As shown in FIG. 6, the minimal diameter of the thread slot 180 can be defined by the circumference of the shaft 130 at a position that defines the thread slot 180, for thread slots 180 that extend to the shaft 130. Alternatively, with reference to FIG. 7, the minimal diameter of the thread slot 180 can be defined by the circumference of the thread 150 at a position that defines the thread slot 180, for thread slots 180 that do not extend to the shaft 130. The maximal diameter of the thread slot 180 can be defined by the thread 150 at a furthest point at which the thread 150 extends radially from the shaft 130. As will be appreciated, the thread slot depth 184 can vary along the orthopedic screw 100, depending for example on dimensions of the shaft 130, the thread 150, and the plurality of grooves 160 along the orthopedic screw 100. For example, the thread slot depth 184 can vary along the orthopedic screw 100 in accordance with loads that the orthopedic screw 100 will need to carry and/or hard tissue with which the orthopedic screw 100 will interface. Also, thread slot depth 184 may differ from thread height 152, e.g. if the thread slots 180 extend into the shaft core 140. In some examples, the thread slots 180 have a thread slot depth 184 of 120 μm to 5,000 μm, 150 μm to 1,000 μm, 200 μm to 800 μm, 250 μm to 600 μm, 300 μm to 500 μm, or about 400 μm. Also in some examples, the thread slots 180 have a thread slot depth 184 of 100 μm to 500 μm, 300 μm to 1,000 μm, 500 μm to 5,000 μm, or 1,000 μm to 10,000 μm.

The orthopedic screw 100 can be made to have thread segments 170 having edge shapes suitable for a specific orthopedic application and/or hard tissue. For example, in some embodiments the thread segments 170 can have an edge shape at a maximal diameter of the thread segments 170, i.e. at a furthest point at which the thread segment 170 extends radially from the shaft 130, corresponding to an acute angle form, e.g. a sharp V-form. This may promote initial implantation of the orthopedic screw 100 into a hard tissue. Also in some examples the thread segments 170 can have an edge shape at a maximal diameter of the thread segments 170 corresponding to a radiused form, e.g. a rounded form. This may avoid irritation to hard tissue during loading. Similarly, in some embodiments the thread segments 170 can have an edge shape at a minimal diameter of the thread segments 170, i.e. at a position at which the thread segment 170 extends radially from the shaft 130, corresponding to an acute angle form. Also in some embodiments the thread segments 170 can have an edge shape at a minimal diameter of the thread segments 170 corresponding to a radiused form. Additional edge shapes, e.g. a blunt form or an irregular form, among others, may also be used. The orthopedic screw 100 also can be made to have grooves 160 having edge shapes suitable for a specific orthopedic application and/or hard tissue, like for the thread segments 170.

In some examples, the orthopedic screw 100 has a plurality of leads, e.g. two, three, or more leads. In other examples, the orthopedic screw 100 has one lead.

As noted, the orthopedic screw 100 has a plurality of grooves 160, i.e. two or more grooves 160. In some examples, the plurality of grooves 160 comprises three or more grooves 160, e.g. 4-6, 7-10, 11-20, 20-40, or more than 40 grooves 160.

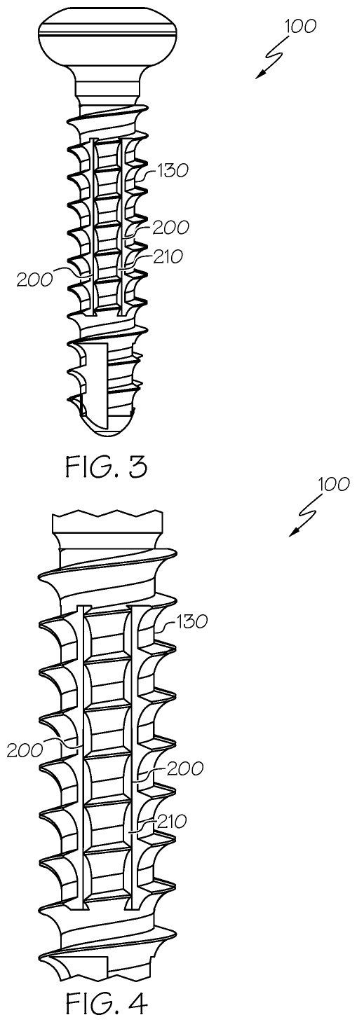

As shown in FIGS. 3-5, in some examples the shaft 130 has a plurality of channels 200 therealong, the plurality of channels 200 corresponding to portions 210 of the plurality of grooves 130 that extend into, and along, the shaft 130.

As shown in FIG. 7, in some examples (including, for example, the trochanteric screw 1001, among others) the shaft 130 further comprises a non-threaded shaft portion 220 between the head 120 and at least one thread 150.

In some examples, the orthopedic screw 100 is self-tapping. As shown in FIG. 1, this can be based, for example, on the orthopedic screw 100 comprising a self-tapping feature 230. In other examples, the orthopedic screw 100 is not self-tapping.

In some examples, the orthopedic screw 100 is self-locking. This can be based, for example, on the orthopedic screw 100 comprising a reverse thread adjacent the head 120, for engagement with a thread adjacent a hole in an implant. In other examples, the orthopedic screw 100 is not self-locking.

As shown in FIG. 1, in some examples, at least one thread 150 comprises a non-grooved thread portion 240 between the tip 110 and the plurality of grooves 160 and/or between the head 120 and the plurality of grooves 160.

The orthopedic screw 100 can be made of one or more materials and/or hard tissues. The one or more materials and/or hard tissues can have a Young's modulus of elasticity, i.e. a tensile modulus of elasticity, of at least 3 GPa, as measured at 21° C. Thus, for example, the orthopedic screw 100 can be made of one or more materials such as implantable-grade polyaryletherketone that is essentially unfilled (such as implantable-grade polyetheretherketone or implantable-grade polyetherketoneketone), titanium, stainless steel, cobalt-chromium alloy, titanium alloy (such as Ti-6A1-4V titanium alloy or Ti-6A1-7Nb titanium alloy), ceramic material (such as silicon nitride (Si3N4)), or implantable-grade composite material (such as implantable-grade polyaryletherketone with filler, implantable-grade polyetheretherketone with filler, implantable-grade polyetheretherketone with carbon fiber, or implantable-grade polyetheretherketone with hydroxyapatite). Specific examples include (i) implantable-grade polyetheretherketone that is essentially unfilled, which has a Young's modulus of approximately 4 GPa, (ii) implantable-grade polyetheretherketone with filler, e.g. carbon-fiber-reinforced implantable-grade polyetheretherketone, which has a Young's modulus of elasticity of at least 18 GPa, (iii) titanium, which has a Young's modulus of elasticity of approximately 110 GPa, (iv) stainless steel, which has a Young's modulus of elasticity of approximately 200 GPa, (v) cobalt-chromium alloy, which has a Young's modulus of elasticity of greater than 200 GPa, or (vi) titanium alloy, which has a Young's modulus of elasticity of approximately 105-120 GPa, all as measured at 21° C. Also for example, the orthopedic screw 100 also can be made of one or more hard tissues such as a hard tissue obtained from a human or animal (such as autologous hard tissue, allogenic hard tissue, or xenogeneic hard tissue), human cartilage, animal cartilage, human bone, animal bone, cadaver bone, or cortical allograft. Such hard tissues obtained from a human or animal can have a Young's modulus of elasticity of, e.g. 4 to 18 GPa. Such hard tissues obtained from a human or animal can also be treated, in advance of implantation, to decrease or eliminate the capacity of the hard tissue to elicit an immune response in an individual upon implantation into the individual. Also for example, the orthopedic screw 100 can be made of one or more materials such as resin for rapid prototyping, SOMOS® NanoTool non-crystalline composite material, SOMOS® 9120 liquid photopolymer, SOMOS® WaterShed XC 11122 resin, ACCURA® XTREME™ White 200 plastic, ACCURA® 60) plastic, or similar material. Also for example, the orthopedic screw 100 can be made of one or more materials matching mechanical properties of bone into which the screw will be implanted, with the one or more materials including one or more of a titanium coating, a ceramic coating, or bone-inducing cpds. Also for example, the orthopedic screw 100 can be made of further combinations of the above-noted materials and/or hard tissues.

As noted, the one or more materials and/or hard tissues of which the orthopedic implant 100 is made can have a Young's modulus of elasticity of at least 3 GPa. Accordingly, the orthopedic screw 100 also can have a Young's modulus of elasticity of at least 3 GPa, for example 18 to 230 GPa, 18 to 25 GPa, 100 to 110 GPa, 190 to 210 GPa, 200 to 230 GPa, 105 to 120 GPa, or 4 to 18 GPa.

The orthopedic screw 100 can be made by methods such as laser cutting, injection molding, 3D printing, and other fabrication methods that are known in the art. In some examples, e.g. for an orthopedic screw 100 made by laser cutting, the thread 150 can be interrupted by grooves 160 according to a regular pattern, such that the thread segments 170 are distributed along the shaft 130 also according to a regular pattern. Also in some examples, e.g. for an orthopedic screw 100 made by 3D printing, the thread 150 can be interrupted by grooves 160 not according to a regular pattern, e.g. randomly, such that the thread segments 170 are distributed along the shaft 130 also not according to a regular pattern, e.g. randomly.

In some examples additional hard tissue can be added to the thread 150 and/or the grooves 160 of the orthopedic screw 100 prior to implanting. For example, shavings of hard tissue of a patient, generated during preparation work including sawing or drilling of hard tissue of the patient, can be added. This may promote growth of tissue into the grooves 160 of the orthopedic screw 100 following implantation.

Also in some examples additional compositions can be added to the thread 150 and/or the grooves 160 of the orthopedic screw 100 prior to implanting. Such compositions include, for example, blood, one or more antibiotics, one or more osteogenic compounds, bone marrow aspirate, and/or surface chemistry for inducing early bone ingrowth. For example, the thread 150 and/or the grooves 160 can be coated with one or more such compositions, with the thread 150 and/or the grooves 160 retaining the compositions during implantation. This also may promote growth of tissue into the grooves 160 of the orthopedic screw 100 following implantation.

With reference to FIGS. 7-11, the trochanteric screw 1001 exemplifies an orthopedic screw 100 in which (1) the head 120 comprises a tool engaging portion 190 for rotationally driving the orthopedic screw 100, (2) the plurality of grooves 160 comprises three or more grooves 160, and (3) the shaft 130 further comprises a non-threaded shaft portion 220 between the head 120 and at least one thread 150.

### Methods of Using Orthopedic Screws

A method of use of the orthopedic screw 100 for fixation of a hard tissue in an individual in need thereof also is disclosed. The orthopedic screw 100 is as described above, including each of the examples as described above.

The method comprises a step of rotationally driving the orthopedic screw 100 into the hard tissue of the individual.

In some examples, the method further comprises, before the step of rotationally driving the orthopedic screw 100, a step of drilling a pilot hole in the hard tissue for the orthopedic screw 100. In accordance with these examples, the orthopedic screw 100 can then be rotationally driven into the pilot hole, for fixation of the hard tissue.

In some embodiments of these examples, the method further comprises, between the step of drilling the pilot hole and the step of rotationally driving the orthopedic screw 100, a step of tapping the pilot hole with a tapping device. In accordance with these embodiments, the orthopedic screw 100 can then be rotationally driven into the tapped pilot hole, for fixation of the hard tissue.

In some examples, the fixation comprises a lag screw technique for applying inter-fragmentary compression within the hard tissue.

In some examples, the fixation comprises a positioning screw technique for holding the hard tissue in a position with respect to another hard tissue.

In some examples, the fixation comprises attaching an implant to the hard tissue.

In some examples the method further comprises, before the step of rotationally driving the orthopedic screw 100, a step of adding additional hard tissue to the thread 150 and/or the grooves 160 of the orthopedic screw 100. Again, for example, shavings of hard tissue of a patient, generated during preparation work including sawing or drilling of hard tissue of the patient, can be added.

Also in some examples the method further comprises, before the step of rotationally driving the orthopedic screw 100, a step of adding additional compositions to the thread 150 and/or the grooves 160 of the orthopedic screw 100 prior to implanting. Again, such compositions can include, for example, blood, one or more antibiotics, one or more osteogenic compounds, bone marrow aspirate, and/or surface chemistry for inducing early bone ingrowth. For example, the step of adding additional compositions can include coating the thread 150 and/or the grooves 160 with one or more such compositions, with the thread 150 and/or the grooves 160 retaining the compositions during implantation.

In some examples, the individual comprises a person having bone with a low bone mass and/or a deteriorated bone structure, due to one or more of age-related osteoporosis, idiopathic primary osteoporosis, secondary osteoporosis, or a neoplastic condition of the musculoskeletal system.

Exemplary hard tissues suitable for this method include bone, cartilage, calcified cartilage, non-calcified cartilage, and tissue that has become mineralized. Exemplary hard tissues also include long bone, maxillary bone, mandibular bone, and membranous bone. Exemplary hard tissues also include tibia, femur, shoulder, small joints, skull, and metatarsal. Exemplary hard tissues also include spine. Exemplary hard tissues also include bones of the foot/ankle in addition to metatarsal, including talus, navicular, cuneiform bones, and phalanges.

### Cannulated Orthopedic Screws

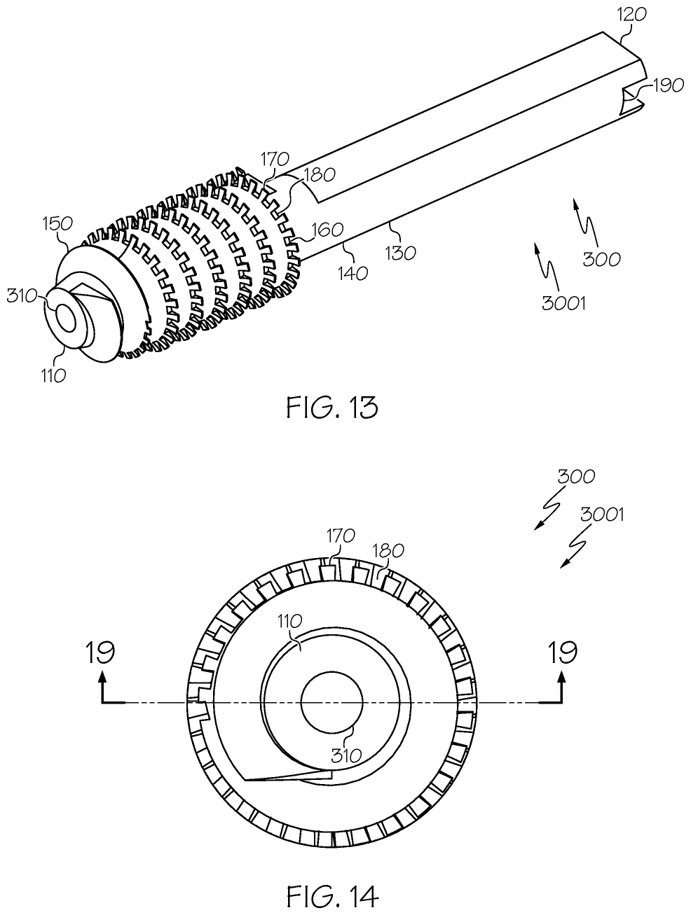

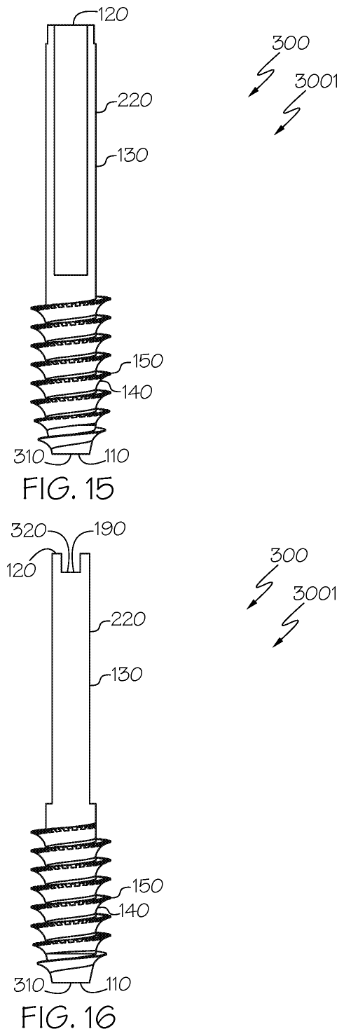

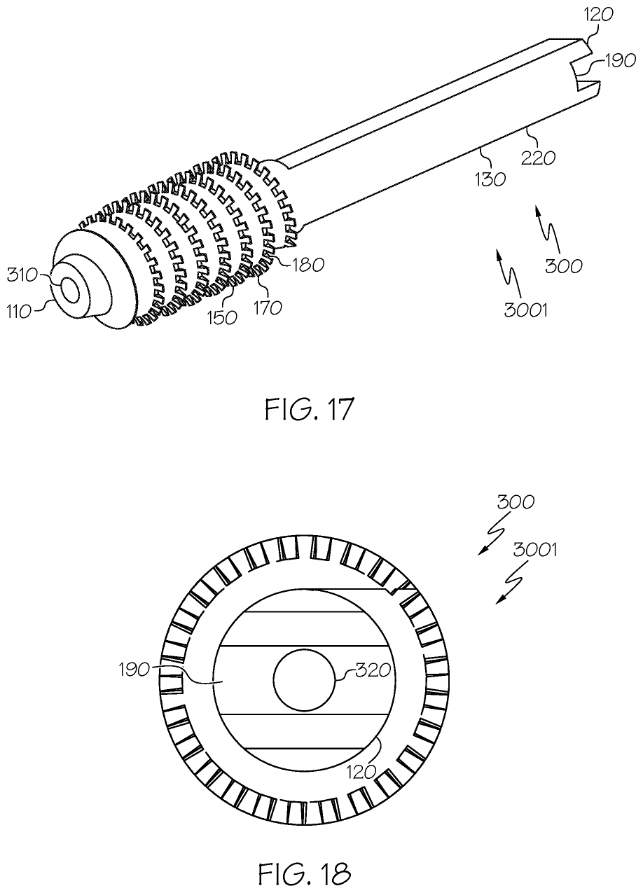

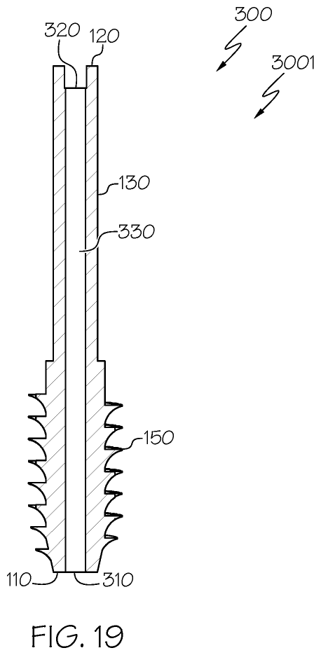

As shown in FIG. 12, also disclosed is a cannulated orthopedic screw 300. FIGS. 13-19 provide illustrations of an example cannulated orthopedic screw 300 corresponding to a cannulated trochanteric screw 3001.

As shown in FIGS. 12-19, the cannulated orthopedic screw 300 includes a tip 110 having a tip opening 310, a head 120 having a head opening 320, and a shaft 130 extending between the tip 110 and the head 120. The shaft 130 comprises a shaft core 140 and at least one thread 150. The at least one thread 150 is disposed helically along the shaft 130, extends radially from the shaft 130, and has a plurality of grooves 160 oriented transversely with respect to the at least one thread 150. The plurality of grooves 160 define a series of thread segments 170 and thread slots 180 along the at least one thread 150. The shaft core 140 is hollow from the tip 110 to the head 120 and defines a lumen 330 extending from the tip opening 310 to the head opening 320.

The tip opening 310, the head opening 320, and the lumen 330 allow the cannulated orthopedic screw 300 to be introduced to a hard tissue of an individual in need thereof over a guide wire or a guide pin because the shaft 130 is hollow from the tip 110 to the head 120.

The cannulated orthopedic screw 300 is otherwise like the orthopedic screw 100 as described above.

Thus, in some examples the cannulated orthopedic screw 300 is tapered from the shaft 130 to the tip 110, whereas in other examples the cannulated orthopedic screw 300 is not so tapered.

In some examples (including, for example, the cannulated trochanteric screw 3001, among others) the head 120 comprises a tool engaging portion 190 for rotationally driving the cannulated orthopedic screw 300. In other examples, the head 120 does not comprise a tool engaging portion.

In some examples at least one thread 150 has a thread height 152 of 100 μm to 10,000 μm, e.g. 120 μm to 5,000 μm, 150 μm to 1,000 μm, 200 μm to 800 μm, 250 μm to 600 μm, 300 μm to 500 μm, or about 400 μm, or e.g. 100 μm to 500 μm, 300 μm to 1,000 μm, 500 μm to 5,000 μm, or 1,000 μm to 10,000 μm.

In some examples, the thread segments 170 have a thread segment width 172 of 100 μm to 10,000 μm, e.g. 120 μm to 5,000 μm, 150 μm to 1,000 μm, 200 μm to 800 μm, 250 μm to 600 μm, 300 μm to 500 μm, or about 400 μm, or e.g. 100 μm to 500 μm, 300 to 1,000 μm, 500 μm to 5,000 μm, or 1,000 μm to 10,000 μm.

In some examples, the thread slots 180 have a thread slot width 182, measured as an arcuate length with respect to the shaft 130, of 100 μm to 10,000 μm, e.g. 120 μm to 5,000 μm, 150 μm to 1,000 μm, 200 μm to 800 μm, 250 μm to 600 μm, 300 μm to 500 μm, or about 400 μm, or e.g. 100 μm to 500 μm, 300 μm to 1,000 μm, 500 μm to 5,000 μm, or 1,000 μm to 10,000 μm.

In some examples, the thread slots 180 have a thread slot depth 184 of 100 μm to 10,000 μm, e.g. 120 μm to 5,000 μm, 150 μm to 1,000 μm, 200 μm to 800 μm, 250 μm to 600 μm, 300 μm to 500 μm, or about 400 μm, or e.g. 100 μm to 500 μm, 300 μm to 1,000 μm, 500 μm to 5,000 μm, or 1,000 μm to 10,000 μm.

In some examples, the cannulated orthopedic screw 300 has a plurality of leads, e.g. two, three, or more leads. In other examples, the cannulated orthopedic screw 300 has one lead.

In some examples, the plurality of grooves 160 comprises three or more grooves 160, e.g. 4-6, 7-10, 11-20, 20-40, or more than 40 grooves 160.

In some examples the shaft 130 has a plurality of channels 200 therealong, the plurality of channels 200 corresponding to portions 210 of the plurality of grooves 130 that extend into, and along, the shaft 130.

In some examples (including, for example, the cannulated trochanteric screw 3001, among others) the shaft 130 further comprises a non-threaded shaft portion 220 between the head 120 and at least one thread 150.

In some examples, the cannulated orthopedic screw 300 is self-tapping, e.g. based on comprising a self-tapping feature 230. In other examples, the cannulated orthopedic screw 300 is not self-tapping.

In some examples, the cannulated orthopedic screw 300 is self-locking, e.g. based on comprising a reverse thread adjacent the head 120. In other examples, the cannulated orthopedic screw 300 is not self-locking.

In some examples, at least one thread 150 comprises a non-grooved thread portion 240 between the tip 110 and the plurality of grooves 160 and/or between the head 120 and the plurality of grooves 160.

The cannulated orthopedic screw 300 can be made of one or more of the materials and/or hard tissues noted above with respect to the orthopedic screw 100. The cannulated orthopedic screw 300 also can have a Young's modulus of elasticity of at least 3 GPa, for example 18 to 230 GPa, 18 to 25 GPa, 100 to 110 GPa, 190 to 210 GPa, 200 to 230 GPa, 105 to 120 GPa, or 4 to 18 GPa.

The cannulated orthopedic screw 300 also can be made by methods such as laser cutting, injection molding, 3D printing, and other fabrication methods that are known in the art. In some examples, the thread 150 can be interrupted by grooves 160 according to a regular pattern, such that the thread segments 170 are distributed along the shaft 130 also according to a regular pattern. Also in some examples, the thread 150 can be interrupted by grooves 160 not according to a regular pattern, e.g. randomly, such that the thread segments 170 are distributed along the shaft 130 also not according to a regular pattern, e.g. randomly.

In some examples, additional hard tissue also can be added to the thread 150 and/or the grooves 160 of the cannulated orthopedic screw 300 prior to implanting.

Also in some examples additional compositions also can be added to the thread 150 and/or the grooves 160 of the cannulated orthopedic screw 300 prior to implanting.

### Methods of Using Cannulated Orthopedic Screws

A method of use of the cannulated orthopedic screw 300 for fixation of a hard tissue in an individual in need thereof also is disclosed. The cannulated orthopedic screw 300 is as described above, including each of the examples as described above.

The method comprises a step of rotationally driving the cannulated orthopedic screw 300 into the hard tissue of the individual.

In some examples, the method further comprises, before the step of rotationally driving the cannulated orthopedic screw 300, a step of drilling a pilot hole in the hard tissue for the cannulated orthopedic screw 300. In accordance with these examples, the cannulated orthopedic screw 300 can then be rotationally driven into the pilot hole, for fixation of the hard tissue.

In some embodiments of these examples, the method further comprises, between the step of drilling the pilot hole and the step of rotationally driving the cannulated orthopedic screw 300, a step of inserting a guide wire or a guide pin into the hard tissue, and a step of introducing the cannulated orthopedic screw 300 to the hard tissue over the guide wire or the guide pin. In accordance with these embodiments, the cannulated orthopedic screw 300 can then be rotationally driven, for fixation of the hard tissue.

In other examples, specific to spine, the method further comprises, before the step of rotationally driving the cannulated orthopedic screw 300, a step of inserting a guide wire or a guide pin into a hard tissue, e.g. a vertebral body, under direct visualization or percutaneously, and a step of tapping over the guide wire or the guide pin. The method can be carried out, for example, as follows: (1) targeting the pedicle percutaneously, for example via fluoroscopic imagining or using a navigation system; (2) inserting a bone biopsy needle, for example a JAMSHIDI™ bone biopsy needle, into the pedicle of the target vertebral level; (3) inserting a guide wire into the vertebral body using the bone biopsy needles as a guide; (4) removing the bone biopsy needle, leaving the guide wire; (5) dilating to provide a channel from the skin to the surface of the bone; (6) tapping to the desired size over the guide wire (and optionally drilling); and (7) inserting the screw over the guide wire into the vertebral body.

In some examples, the fixation comprises a lag screw technique for applying inter-fragmentary compression within the hard tissue.

In some examples, the fixation comprises a positioning screw technique for holding the hard tissue in a position with respect to another hard tissue.

In some examples, the fixation comprises attaching an implant to the hard tissue.

In some examples the method further comprises, before the step of rotationally driving the cannulated orthopedic screw 300, a step of adding additional hard tissue to the thread 150 and/or the grooves 160 of the cannulated orthopedic screw 300.

Also in some examples the method further comprises, before the step of rotationally driving the cannulated orthopedic screw 300, a step of adding additional compositions to the thread 150 and/or the grooves 160 of the cannulated orthopedic screw 300 prior to implanting.

In some examples, the individual comprises a person having bone with a low bone mass and/or a deteriorated bone structure, due to one or more of age-related osteoporosis, idiopathic primary osteoporosis, secondary osteoporosis, or a neoplastic condition of the musculoskeletal system.

Exemplary hard tissues suitable for this method include bone, cartilage, calcified cartilage, non-calcified cartilage, and tissue that has become mineralized. Exemplary hard tissues also include long bone, maxillary bone, mandibular bone, and membranous bone. Exemplary hard tissues also include tibia, femur, shoulder, small joints, skull, and metatarsal. Exemplary hard tissues also include spine. Exemplary hard tissues also include bones of the foot/ankle in addition to metatarsal, including talus, navicular, cuneiform bones, and phalanges.

### Exemplary Embodiments

The following are exemplary embodiments of the hard-tissue stem implant, the method of making the hard-tissue stem implant, and the method of use of the hard-tissue stem implant as disclosed herein.

Embodiment A: An orthopedic screw comprising:

(a) a tip;

(b) a head; and

(c) a shaft extending between the tip and the head,

wherein:

(i) the shaft comprises a shaft core and at least one thread;

(ii) the at least one thread is disposed helically along the shaft, extends radially from the shaft, and has a plurality of grooves oriented transversely with respect to the at least one thread;

(iii) the plurality of grooves define a series of thread segments and thread slots along the at least one thread; and

(iv) the shaft core is solid from the tip to the head.

Embodiment B: The orthopedic screw of embodiment A, wherein the orthopedic screw is tapered from the shaft to the tip.

Embodiment C: The orthopedic screw of embodiment A or embodiment B, wherein the head comprises a tool engaging portion for rotationally driving the orthopedic screw.

Embodiment D: The orthopedic screw of any one of embodiments A-C, wherein the at least one thread has a thread height of 100 μm to 10,000 μm.

Embodiment E: The orthopedic screw of any one of embodiments A-D, wherein the thread segments have a thread segment width, measured as an arcuate length with respect to the shaft, of 100 μm to 10,000 μm.

Embodiment F: The orthopedic screw of any one of embodiments A-E, wherein the thread slots have a thread slot width, measured as an arcuate length with respect to the shaft, of 100 μm to 10,000 μm.

Embodiment G: The orthopedic screw of any one of embodiments A-F, wherein the thread slots have a thread slot depth of 100 μm to 10,000 μm

Embodiment H: The orthopedic screw of any one of embodiments A-G, wherein the orthopedic screw has a plurality of leads.

Embodiment I: The orthopedic screw of any one of embodiments A-H, wherein the plurality of grooves comprises three or more grooves.

Embodiment J: The orthopedic screw of any one of embodiments A-I, wherein the shaft has a plurality of channels therealong, the plurality of channels corresponding to portions of the plurality of grooves that extend into, and along, the shaft.

Embodiment K: The orthopedic screw of any one of embodiments A-J, wherein the shaft further comprises a non-threaded shaft portion between the head and the at least one thread.

Embodiment L: The orthopedic screw of any one of embodiments A-K, wherein the orthopedic screw is self-tapping.

Embodiment M: The orthopedic screw of any one of embodiments A-L, wherein the orthopedic screw is self-locking.

Embodiment N: The orthopedic screw of any one of embodiments A-M, wherein the at least one thread comprises a non-grooved thread portion between the tip and the plurality of grooves and/or between the head and the plurality of grooves.

Embodiment O: The orthopedic screw of any one of embodiments A-N, wherein the orthopedic screw is made of one or more materials selected from implantable-grade polyaryletherketone that is essentially unfilled, implantable-grade polyetheretherketone, implantable-grade polyetherketoneketone, titanium, stainless steel, cobalt-chromium alloy, titanium alloy, Ti-6A1-4V titanium alloy, Ti-6A1-7Nb titanium alloy, ceramic material, silicon nitride (Si3N4), implantable-grade composite material, implantable-grade polyaryletherketone with filler, implantable-grade polyetheretherketone with filler, implantable-grade polyetheretherketone with carbon fiber, or implantable-grade polyetheretherketone with hydroxyapatite.

Embodiment P: The orthopedic screw of any one of embodiments A-N, wherein the orthopedic screw is made of one or more materials selected from resin for rapid prototyping, SOMOS® NanoTool non-crystalline composite material, SOMOS® 9120 liquid photopolymer, SOMOS® WaterShed XC 11122 resin, ACCURA® XTREME™ White 200 plastic, ACCURA® 60) plastic, or similar material.

Embodiment Q: The orthopedic screw of any one of embodiments A-N, wherein the orthopedic screw is made of one or more materials matching mechanical properties of bone into which the screw will be implanted, with the one or more materials including one or more of a titanium coating, a ceramic coating, or bone-inducing cpds.

Embodiment R: A method of use of the orthopedic screw of any one of embodiments A-Q for fixation of a hard tissue in an individual in need thereof, the method comprising a step of rotationally driving the orthopedic screw into the hard tissue of the individual.

Embodiment S: The method of embodiment R, the method further comprising, before the step of rotationally driving the orthopedic screw, a step of drilling a pilot hole in the hard tissue for the orthopedic screw.

Embodiment T: The method of embodiment S, the method further comprising, between the step of drilling the pilot hole and the step of rotationally driving the orthopedic screw, a step of tapping the pilot hole with a tapping device.

Embodiment U: The method of any one of embodiments R-T, wherein the fixation comprises a lag screw technique for applying inter-fragmentary compression within the hard tissue.

Embodiment V: The method of any one of embodiments R-T, wherein the fixation comprises a positioning screw technique for holding the hard tissue in a position with respect to another hard tissue.

Embodiment W: The method of any one of embodiments R-T, wherein the fixation comprises attaching an implant to the hard tissue.

Embodiment X: The method of any one of embodiments R-W, wherein the individual comprises a person having bone with a low bone mass and/or a deteriorated bone structure due to one or more of age-related osteoporosis, idiopathic primary osteoporosis, secondary osteoporosis, or a neoplastic condition of the musculoskeletal system.

Embodiment AA: A cannulated orthopedic screw comprising:

(a) a tip having a tip opening;

(b) a head having a head opening; and

(c) a shaft extending between the tip and the head; and

wherein:

(i) the shaft comprises a shaft core and at least one thread;

(ii) the at least one thread is disposed helically along the shaft, extends radially from the shaft, and has a plurality of grooves oriented transversely with respect to the at least one thread;

(iii) the plurality of grooves define a series of thread segments and thread slots along the at least one thread; and

(iv) the shaft core is hollow from the tip to the head and defines a lumen extending from the tip opening to the head opening.

Embodiment BB: The cannulated orthopedic screw of embodiment AA, wherein the cannulated orthopedic screw is tapered from the shaft to the tip.

Embodiment CC: The cannulated orthopedic screw of embodiment AA or embodiment BB, wherein the head comprises a tool engaging portion for rotationally driving the cannulated orthopedic screw.

Embodiment DD: The cannulated orthopedic screw of any one of embodiments AA-CC, wherein the at least one thread has a thread height of 100 μm to 10,000 μm.

Embodiment EE: The cannulated orthopedic screw of any one of embodiments AA-DD, wherein the thread segments have a thread segment width, measured as an arcuate length with respect to the shaft, of 100 μm to 10,000 μm.

Embodiment FF: The cannulated orthopedic screw of any one of embodiments AA-EE, wherein the thread slots have a thread slot width, measured as an arcuate length with respect to the shaft, of 100 μm to 10,000 μm.

Embodiment GG: The cannulated orthopedic screw of any one of embodiments AA-FF, wherein the thread slots have a thread slot depth of 100 μm to 10,000 μm.

Embodiment HH: The cannulated orthopedic screw of any one of embodiments AA-GG, wherein the cannulated orthopedic screw has a plurality of leads.

Embodiment II: The cannulated orthopedic screw of any one of embodiments AA-HH, wherein the plurality of grooves comprises three or more grooves.

Embodiment JJ: The cannulated orthopedic screw of any one of embodiments AA-II, wherein the shaft has a plurality of channels therealong, the plurality of channels corresponding to portions of the plurality of grooves that extend into, and along, the shaft.

Embodiment KK: The cannulated orthopedic screw of any one of embodiments AA-JJ, wherein the shaft further comprises a non-threaded shaft portion between the head and the at least one thread.

Embodiment LL: The cannulated orthopedic screw of any one of embodiments AA-KK, wherein the cannulated orthopedic screw is self-tapping.

Embodiment MM: The cannulated orthopedic screw of any one of embodiments AA-LL, wherein the cannulated orthopedic screw is self-locking.

Embodiment NN: The cannulated orthopedic screw of any one of embodiments AA-MM, wherein the at least one thread comprises a non-grooved thread portion between the tip and the plurality of grooves and/or between the head and the plurality of grooves.

Embodiment OO: The cannulated orthopedic screw of any one of embodiments AA-NN, wherein the cannulated orthopedic screw is made of one or more materials selected from implantable-grade polyaryletherketone that is essentially unfilled, implantable-grade polyetheretherketone, implantable-grade polyetherketoneketone, titanium, stainless steel, cobalt-chromium alloy, titanium alloy, Ti-6A1-4V titanium alloy, Ti-6A1-7Nb titanium alloy, ceramic material, silicon nitride (Si3N4), implantable-grade composite material, implantable-grade polyaryletherketone with filler, implantable-grade polyetheretherketone with filler, implantable-grade polyetheretherketone with carbon fiber, or implantable-grade polyetheretherketone with hydroxyapatite.

Embodiment PP: The cannulated orthopedic screw of any one of embodiments AA-NN, wherein the cannulated orthopedic screw is made of one or more materials selected from resin for rapid prototyping, SOMOS® NanoTool non-crystalline composite material, SOMOS® 9120 liquid photopolymer, SOMOS® WaterShed XC 11122 resin, ACCURA® XTREME™ White 200 plastic, ACCURA® 60) plastic or similar material.

Embodiment QQ: The cannulated orthopedic screw of any one of embodiments AA-NN, wherein the cannulated orthopedic screw is made of one or more materials matching mechanical properties of bone into which the screw will be implanted, with the one or more materials including one or more of a titanium coating, a ceramic coating, or bone-inducing cpds.

Embodiment RR: A method of use of the cannulated orthopedic screw of any one of embodiments AA-QQ for fixation of a hard tissue in an individual in need thereof, the method comprising a step of rotationally driving the cannulated orthopedic screw into the hard tissue of the individual.

Embodiment SS: The method of embodiment RR, the method further comprising, before the step of rotationally driving the cannulated orthopedic screw, a step of drilling a pilot hole in the hard tissue for the cannulated orthopedic screw.

Embodiment TT: The method of embodiment SS, the method further comprising, between the step of drilling the pilot hole and the step of rotationally driving the cannulated orthopedic screw, a step of inserting a guide wire or a guide pin into the hard tissue, and a step of introducing the cannulated orthopedic screw to the hard tissue over the guide wire or the guide pin.

Embodiment UU: The method of any one of embodiments RR-TT, wherein the fixation comprises a lag screw technique for applying inter-fragmentary compression within the hard tissue.

Embodiment VV: The method of any one of embodiments RR-TT, wherein the fixation comprises a positioning screw technique for holding the hard tissue in a position with respect to another hard tissue.

Embodiment WW: The method of any one of embodiments RR-TT, wherein the fixation comprises attaching an implant to the hard tissue.

Embodiment XX: The method of any one of embodiments RR-WW, wherein the individual comprises a person having bone with a low bone mass and/or a deteriorated bone structure due to one or more of age-related osteoporosis, idiopathic primary osteoporosis, secondary osteoporosis, or a neoplastic condition of the musculoskeletal system.

It will be apparent to those skilled in the art that various modifications and variations can be made without departing from the spirit and scope of the claimed invention.

1-17. (canceled)

18. A method of use of an orthopedic screw for fixation of a hard tissue in an individual in need thereof, the screw comprising:
(a) a tip;
(b) a head; and
(c) a shaft extending between the tip and the head,
wherein:
(i) the shaft comprises a shaft core and at least one thread;
(ii) the at least one thread is disposed helically along the shaft, extends radially from the shaft, and has a plurality of grooves oriented transversely with respect to the at least one thread;
(iii) the plurality of grooves define a series of thread segments and thread slots along the at least one thread; and
(iv) the shaft core is solid from the tip to the head,
the method comprising a step of rotationally driving the orthopedic screw into the hard tissue of the individual.

19. The method of claim 18, the method further comprising, before the step of rotationally driving the orthopedic screw, a step of drilling a pilot hole in the hard tissue for the orthopedic screw.

20. The method of claim 19, the method further comprising, between the step of drilling the pilot hole and the step of rotationally driving the orthopedic screw, a step of tapping the pilot hole with a tapping device.

21. The method of claim 18, wherein the fixation comprises a lag screw technique for applying inter-fragmentary compression within the hard tissue.

22. The method of claim 18, wherein the fixation comprises a positioning screw technique for holding the hard tissue in a position with respect to another hard tissue.

23. The method of claim 18, wherein the fixation comprises attaching an implant to the hard tissue.

24. The method of claim 18, wherein the individual comprises a person having bone with a low bone mass and/or a deteriorated bone structure due to one or more of age-related osteoporosis, idiopathic primary osteoporosis, secondary osteoporosis, or a neoplastic condition of the musculoskeletal system.

25-41. (canceled)

42. A method of use of a cannulated orthopedic screw for fixation of a hard tissue in an individual in need thereof, the screw comprising
(a) a tip having a tip opening;
(b) a head having a head opening; and
(c) a shaft extending between the tip and the head; and
wherein:
(i) the shaft comprises a shaft core and at least one thread;
(ii) the at least one thread is disposed helically along the shaft, extends radially from the shaft, and has a plurality of grooves oriented transversely with respect to the at least one thread;
(iii) the plurality of grooves define a series of thread segments and thread slots along the at least one thread; and
(iv) the shaft core is hollow from the tip to the head and defines a lumen extending from the tip opening to the head opening,
the method comprising a step of rotationally driving the cannulated orthopedic screw into the hard tissue of the individual.

43. The method of claim 42, the method further comprising, before the step of rotationally driving the cannulated orthopedic screw, a step of drilling a pilot hole in the hard tissue for the cannulated orthopedic screw.

44. The method of claim 43, the method further comprising, between the step of drilling the pilot hole and the step of rotationally driving the cannulated orthopedic screw, a step of inserting a guide wire or a guide pin into the hard tissue, and a step of introducing the cannulated orthopedic screw to the hard tissue over the guide wire or the guide pin.

45. The method of claim 42, wherein the fixation comprises a lag screw technique for applying inter-fragmentary compression within the hard tissue.

46. The method of claim 42, wherein the fixation comprises a positioning screw technique for holding the hard tissue in a position with respect to another hard tissue.

47. The method of claim 42, wherein the fixation comprises attaching an implant to the hard tissue.

48. The method of claim 42, wherein the individual comprises a person having bone with a low bone mass and/or a deteriorated bone structure due to one or more of age-related osteoporosis, idiopathic primary osteoporosis, secondary osteoporosis, or a neoplastic condition of the musculoskeletal system.

49. The method of claim 18, wherein the thread segments have a thread segment width, measured as an arcuate length with respect to the shaft, of 100 μm to 10,000 μm.

50. The method of claim 18, wherein the thread slots have a thread slot width, measured as an arcuate length with respect to the shaft, of 100 μm to 10,000 μm.

51. The method of claim 18, wherein the shaft has a plurality of channels therealong, the plurality of channels corresponding to portions of the plurality of grooves that extend into, and along, the shaft.

52. The method of claim 18, wherein the at least one thread comprises a non-grooved thread portion between the tip and the plurality of grooves and/or between the head and the plurality of grooves.

53. The method of claim 42, wherein the thread segments have a thread segment width, measured as an arcuate length with respect to the shaft, of 100 μm to 10,000 μm.

54. The method of claim 42, wherein the thread slots have a thread slot width, measured as an arcuate length with respect to the shaft, of 100 μm to 10,000 μm.

55. The method of claim 42, wherein the shaft has a plurality of channels therealong, the plurality of channels corresponding to portions of the plurality of grooves that extend into, and along, the shaft.

56. The method of claim 42, wherein the at least one thread comprises a non-grooved thread portion between the tip and the plurality of grooves and/or between the head and the plurality of grooves.
